(19) United States
(12) Patent Application Publication
(10) Pub. No.: US 2024/0415747 A1
(43) Pub. Date: Dec. 19, 2024

(54) IMPROVED FORMULATIONS OF PERSONAL CARE PRODUCTS FOR MASKING ODOR OR REDUCING PERSPIRATION

(71) Applicant: CARGILL, INCORPORATED, Wayzata, MN (US)

(72) Inventors: Nabila BELHAJ, Saint Cloud (FR); Dominique Karine CLIO, Oplinter (BE); Verdu Benoit Stephane NORBERT, Mazamet (FR)

(73) Assignee: CARGILL, INCORPORATED, Wayzata, MN (US)

(21) Appl. No.: 18/704,746

(22) PCT Filed: Nov. 10, 2022
(86) PCT No.: PCT/US2022/079594

§ 371 Date: Apr. 25, 2024

Related U.S. Application Data
(60) Provisional application No. 63/263,846, filed on Nov. 10, 2021.

(51) Int. Cl. A61K 8/26; A61K 8/73; A61Q 15/00

(52) U.S. Cl. CPC A61K 8/26; A61K 8/73; A61K 8/732; A61Q 15/00

(57) ABSTRACT

The present disclosure relates to a personal care formulation for masking odor or reducing perspiration comprising a texturizer, a 1,3-β-D-glucan, and an active ingredient wherein: the texturizer is a mixture of hydroxypropylated starch and nOSA starch; and the active ingredient is selected from the group consisting of fragrances and anti-perspirant actives or a combination thereof.

### CROSS-REFERENCE TO RELATED APPLICATIONS

This application claims the benefit of U.S. Provisional Application No. 63/263,846, filed Nov. 10, 2021, which is incorporated by reference herein in its entirety.

### FIELD OF INVENTION

The present disclosure relates to the use of modified starches and hydrocolloids for use in personal care applications. Specifically, the use of a blend of hydroxypropyl modified and n-octanlysuccinate modified starches in combination with a 1,3-β-D-glucan such as sclaroglucan and an active ingredient. Such a combination allows for the manufacturing of improved more naturally sourced personal care products including antiperspirant and deodorant formulations.

### BACKGROUND

Deodorant and antiperspirant products come in different products formulation and delivery systems. Most typically they are sold as sprays, sticks, or roll-on formulations. Consumers will prefer or choose one of these alternatives for a variety of personal reasons such perceived effectiveness or convenience of use.

Conventional roll-on products used as antiperspirants and/or deodorants are usually emulsions or anhydrous suspensions. Emulsions can be oil-in-water or water-in-oil systems where one phase is dispersed in the other. In emulsions both phases remain together because of the addition of surfactants. Emulsified products tend to have a wet feel, a higher level of tackiness and some irritation issues. Anhydrous suspensions are composed of powders suspended in anhydrous liquids. They typically include a relatively high percentage of suspending agents that contribute to white residues being left on skin and clothing. While anhydrous suspensions do not produce a wet feel, they exhibit other problems such as settling of the powder over time and leakage of the carrier liquid, especially if the product is not continuously shaken.

The present invention reduces and/or eliminates some of the aforementioned problems. In particular, the more environmentally friendly compositions of the present disclosure are able to provide antiperspirants and/or deodorants which have improved efficacy, reduction in the presence of irritating surfactants, reduced white residue on skin and clothing, and improved aesthetics such as quicker drying, faster film formation, and reduced wetness.

### BRIEF DESCRIPTION OF THE FIGURE

FIG. 1 shows the comparison of the speed of film formation of the Examples of the present invention as compared to two commercial benchmarks. The Examples clearly demonstrate dramatically improved ability to create film vs the benchmarks.

### SUMMARY OF INVENTION

Surprisingly, the applicants have observed that the combination of a blend of modified starches and a 1,3-β-D-glucan together can provide favorable viscosity and texture to personal care formulations.

Herein is disclosed and described a personal care formulation for masking odor or reducing perspiration comprising a texturizer, a 1,3-β-D-glucan, and an active ingredient wherein: the texturizer is a mixture of hydroxypropylated starch and nOSA starch; and the active ingredient is selected from the group consisting of fragrances and anti-perspirant actives or a combination thereof.

In some aspects, the 1,3-β-D-glucan is a sclaroglucan and the active ingredient is an antiperspirant active aluminum salt.

In some aspects, the personal care formulation further comprises a silicone.

### DETAILED DESCRIPTION

While personal care formulations are designed for specific purposes, certain considerations are commonly important to most every formulation. Namely, stability, texture, and viscosity are essential to most every personal care formulation.

Stability is the resistance of a formulation to separate into its separate components over time. Stability of a formulation is a critical consideration for both manufacturers and consumers. Personal care products with poor stability are almost universally rejected by consumers and therefore not of commercial interest. Often personal care formulations contain multiple ingredients and are complex mixtures including colorants, pearlescent, conditioning agents, and other components that need to be held in suspension over long periods of time. Stability of a formulation avoids both settling during shipping and storing as well as the need of a customer to shake a formulation prior to use to re-suspend the ingredients.

Texture of a personal care formulation is an important aspect of the consumer experience. Texture is important at multiple times during the user experience. Initially the consumer experiences the texture of a personal care product as it is dispensed (from the bottle to the hand or hair), then the texture is again experienced as the product is used. For example, as a deodorant or anti-perspirant is dispensed its texture (such as smoothness) is viewed by the consumer then as the product dries it is once again perceived by the consumer. Therefore, texture may be evaluated with parameters such as product smoothness, film formation, dryness, and drying over time.

Viscosity is a parameter used to evaluate the thickness and resistance to flow of a material. Proper viscosity is relevant to personal care formulations when dispensing and utilizing a product. If a product has a low viscosity it dispenses too quickly and spreads unevenly when used. If a product has a high viscosity it may be difficult to dispense and spread upon use.

The ability of an antiperspirant/deodorant formulation to create a film on the skin is a critically important aspect to its overall effectiveness and acceptability by the consumer. Use of the product is tied directly to this parameter as consumers desire to quickly dispense an appropriate amount of product during use. In addition, the ability of the product to dry quickly is the next essential aspect perceived by the consumer. Quick drying eliminates or reduces the transfer of product to the users clothes and provide for a more comfortable experience by avoiding sensations such as wetness or tackiness.

The personal care formulations disclosed herein surprisingly have an excellent combination of texture, stability, film formation, drying, and viscosity. Formulations have extended shelf stability even at elevated temperatures as well as desirable viscosity. In addition, the formulations disclosed herein are quick to form films and dry after application. These properties are important factors for deodorants and antiperspirants.

### Starches

Starch is a polymeric carbohydrate consisting of a large number of glucose units joined by glycosidic bonds. This polysaccharide is produced by most green plants as a means to store energy. It is the most common carbohydrate in human diets and is contained in large amounts in staple foods. Starches useful in the present disclosure can come from any plant source including but not limited to: potatoes, wheat, maize (com), rice, tapioca, quinoa, cassava, and the like. Pure starch is a white, tasteless and odorless powder that is insoluble in cold water or alcohol. It consists of two types of molecules: the linear and helical amylose and the branched amylopectin. Depending on the plant, starch generally contains 20 to 25% amylose and 75 to 80% amylopectin by weight. Waxy corn starches contain over 99% amylopectin. When it is isolated directly from the plant source it is most often referred to as “native starch”. Native starch requires heat to thicken or gelatinize. When a starch is pre-cooked, it can then be used to thicken instantly in cold water. This is referred to as a pregelatinized starch.

Native starch can be hydrolyzed into simpler carbohydrates by temperature, acids, various enzymes, or a combination of the three. The resulting fragments are known as dextrins or hydrolyzed starch. The extent of conversion is typically quantified by dextrose equivalent (DE), which is roughly the fraction of the glycosidic bonds in starch that have been broken. For example, maltodextrin is a lightly hydrolyzed (DE 10-20) starch product used as a bland-tasting filler and thickener. Various glucose syrups (DE 30-70), also called corn syrups in the US, are a type of hydrolyzed starch that are viscous solutions used as sweeteners and thickeners in many kinds of processed foods. Dextrose (DE 100), commercial glucose, is prepared by the complete hydrolysis of starch.

A modified starch has a structure that has been altered from its native state, resulting in modification of one or more of its chemical or physical properties. Starches may be modified, for example, by enzymes, oxidation or, substitution with various compounds. For example, starches can be modified to increase stability against heat, acids, or freezing, improve texture, increase or decrease viscosity, increase or decrease gelatinization times, and/or increase or decrease solubility, among others. Modified starches may be partially or completely degraded into shorter chains or glucose molecules. Amylopectin may be debranched. In one example, modified starches are cross-linked for example to improve stability. Starches that are modified by substitution have a different chemical composition.

The base-starch used as the base material for obtaining the modified-starch utilized in the present invention can be sourced from any starch containing sources (hereinafter base material) including for example corn, wheat, potato, tapioca, barley, pea, dent corn, waxy maize, sago, rice, sorghum and high amylose starch, i.e., starch having at least 45% and more particularly at least 65% amylose content, such as high amylose corn. Starch flours may also be used.

The base-starch may be chemically converted, enzymatically converted, or converted by heat treatment or by physical treatment. The term “chemically converted” or “chemical conversion” include, but is not limited to crosslinking, modification with blocking groups to inhibit retrogradation, modification by the addition of lipophilic groups, acetylated starches, hydroxyethylated and hydroxypropylated starches, inorganically esterified starches, cationic, anionic and oxidized starches, zwitterionic starches and combinations thereof. By an “enzymatically converted starch” is herein understood starches converted by enzymes. Heat treatment includes for example pre-gelatinization. The base-starch may have a granular state, which is preferred, or a non-granular state, i.e., the granular state of the starch has been disrupted by physical, thermal, chemical or enzymatic treatment. Preferred base materials include converted or non-converted starches originating from corn, high amylose corn, wheat, potato, tapioca, waxy maize, sago or rice. In some aspects, base materials to manufacture the base-starches are those chosen from the group consisting of corn starch, wheat starch and potato starch. In some aspects, materials are corn starch and wheat starch.

In some aspects, the base-starch is chosen from the group consisting of maltodextrins; pyrodextrins; dextrins such as those prepared by hydrolytic action of acid and/or heat or by the action of enzymes; degraded starches such as for example fluidity or thin boiled starches prepared for example by enzyme conversion, thermal treatment or acid hydrolysis; oxidized starches prepared by treatment with oxidants such as sodium hypochlorite, peroxides and persulfates; and derivatized starches such as cationic, anionic, amphoteric, non-ionic and cross-linked. Any base material can be used for producing these base-starches, such as for example those mentioned above.

In some aspects, the base-starch is chosen from the group consisting of maltodextrins, dextrins, thin boiled starches and oxidized starches, said base-starch being produced from a base material chosen from the group consisting of non-converted corn starch, non-converted wheat starch and non-converted potato starch.

In some aspects, the base-starch is chosen from the group consisting of maltodextrins, dextrins, thin boiled starches, and oxidized starches, said base-starch being produced from a base material those chosen from the group consisting of non-converted corn starch and non-converted wheat starch.

A hydroxypropylated starch (HP starch) is another example of a modified starch that has been functionalized by hydroxypropylation. Such hydroxypropylated starches are well known in the art and are “E-coded” under the designation 1400 in the International System for Food Additives (INS). The hydroxypropylated starches may be prepared from waxy maize starch. In some aspects, hydroxypropylated starches of the present invention include C*HiForm™ 12748 commercially available from Cargill Incorporated. A nOSA starch is a modified starch that has been partially substituted, e.g., from about 0.1% to about 3%, with n-octenyl succinic anhydride. Preferred nOSA starches of the present invention include C*EmTex 12688 (sodium starch octenylsuccinate) commercially available from Cargill Incorporated.

### Blend

As used herein, the term “blend” refers to a physical mixture of two or more substances. An alternative embodiment of the present invention comprises a blend of starches useful for preparations of the emulsions described herein. Advantageously, the emulsions of the present invention are manufactured using a blend comprising or consisting of sodium starch octenylsuccinate and hydroxypropyl starch phosphate. HP starch can be present in the blend in an amount of 5-25%. In one aspect, HP is present in an amount of 15-25% in the blend. In one aspect, nOSA can present in the blend in an amount of 75-95%. In one aspect nOSA is present in an amount of 75-85% in the blend. In one aspect, HP starch is present in the blend in an amount of 15-25% and nOSA starch is present in the blend in an amount of 75-85%

The starch blend and additionally contain other dry ingredients if desired in the final emulsions such at texturizers, colorants, favors, fragrances, preservatives, and the like.

The blend can be prepared according to blending methods for dry ingredients that are known in the art. Use of a standard mixing equipment for a sufficient time will easily prepare the blends described herein.

Such starch blends are commercially available from Cargill Incorporated under the brand Star Design Power and are further described in WO2020/190697 which is hereby incorporated by reference.

The starch blend may be included in the final personal care formulation in an effective amount to create the viscosity and texture required by the particular application. In some aspects, the starch blend is present at between 1 and 10%. In other aspects the starch blend is present at between 2 and 5%.

### 1,3-β-D-Glucan

1,3-β-D-glucan is a polysaccharide characterized by a backbone of D-glucose residues linked in β-(1,3) fashion, wherein the different 1,3-β-D-glucan is structurally differ from each other in terms of their side groups and molecular weights. Curdlan for example, consists exclusively of the β-(1,3)-D glucose backbone, whereas schizophyllan, scleroglucan and yeast glucan contain β-(1,6)-glycosyl side chains. 1,3-β-D-glucan are typically produced by microbial fermentation, the fermentation broth being used directly or in diluted or purified form, usually after having been pasteurized (see e.g., U.S. Pat. No. 3,301,848). The 1,3-β-D-glucan can be used in purified form or as a mixture of 1,3-β-D-glucan and fermentation residuals. For the purpose of the invention, the 1,3-β-D-glucan used is preferably purified to reduce and neutralize the amount and activity of microbial cells and/or water-soluble constituents of the fermentation broth other than the 1,3-β-D-glucan. WO 2009/062561 discloses a method of manufacturing such high purity 1,3-β-D-glucans.

The 1,3-β-D-glucans used within the present invention include any polysaccharides classified as 1,3-β-D-glucans, i.e., any polysaccharide which has β-(1,3)-linked backbone of D-glucose residues. Examples of such 1,3-β-D-glucans include curdlan (a homopolymer of β-(1,3)-linked D-glucose residues produced from, e.g., Agrobacterium spp.), grifolan (a branched 1,3-β-D-glucan produced from, e.g., the fungus Grifola frondosa), lentinan (a branched 1,3-β-D-glucan having two glucose branches attached at each fifth glucose residue of β-(1,3)-backbone produces from, e.g., the fungus Lentinus eeodes), schizophyllan (a branched 1,3-β-D-glucan having one glucose branch for every third glucose residue in the β-(1,3)-backbone produced from, e.g., the fungus Schizophyllan commune), scleroglucan (a branched 1,3-β-D-glucan with one out of three glucose molecules of the β-(1,3)-backbone being linked to a side D-glucose unit by a (1,6)-β bond produced from, e.g., fungi of the Sclerotium spp.), SSG (a highly branched β-(1,3)-glucan produced from, e.g., the fungus Sclerotinia sclerotiorum), soluble glucans from yeast (a 1,3-β-D-glucan with β-(1-6)-linked side groups produced from, e.g., Saccharomyces cerevisiae), and laminarin (a 1,3-β-D-glucan with β-(1,3)-glucan and β-(1,6)-glucan side groups produced from, e.g., the brown algae Laminaria digitata).

In some aspects, the 1,3-β-D-glucan is scleroglucan.

Scleroglucan is a natural polysaccharide produced by fermentation of the filamentous fungus Sclerotium rolfsii. Its chemical structure consists of a linear β(1-3) D-glucose backbone with one β(1-6) D-glucose side chain every three main residue, as shown below:

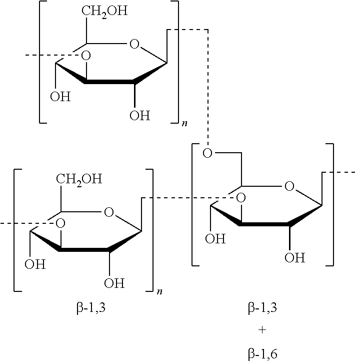

Scleroglucan exhibits high potential for commercialization and may show different branching frequency, side-chain length, and/or molecular weight depending on the producing strain or culture conditions. As used herein, the term “molecular weight” refers to the measure of the sum of the atomic weight values of the atoms in a molecule.

When the 1,3-β-D-glucan used is scleroglucan, it may be in a purified form or in a mix with fermentation residuals.

Scleroglucan may be in the form of native scleroglucan or hydrolysed scleroglucan. The molecular weight of the scleroglucan may be between 50,000 and 6,000,000 Daltons. Preferably, if the scleroglucan is native scleroglucan, the molecular weight of the scleroglucan may be between 1,500,000 and 6,000,000 Daltons. In one aspect, the scleroglucan is a hydrolysed scleroglucan, the molecular weight may be between 50,000 and 100,000 Dalton.

Scleroglucan is able to from a triple helix in solution making it an ideal nature-derived thickener and stabilizer for formulations with smooth and soft textures. Water-solubility, viscosifying ability and wide stability over temperature, pH and salinity make scleroglucan useful for different personal care applications.

Various types of 1,3-β-D-glucans are commercially available from numerous sources including Cargill Incorporated under the brand name Actigum and specifically Actigum CS11.

The 1,3-β-D-glucans may be included in the final personal care formulation in an effective amount to create the viscosity and texture required by the particular application. In some aspects, the 1,3-β-D-glucans is present at between 0.1 and 5%. In other aspects the starch blend is present at between 0.25 and 5%.

### Topical Formulation

The combination provided herein is useful in the manufacture of topical formulations such as personal care products. The inventors unexpectedly found that formulations comprising a both a blend of modified starches and a 1,3-β-D-glucan have numerous desirable characteristics as explained further below.

In one aspect, the present invention is a topical formulation comprising a blend of modified starches and a 1,3-β-D-glucan as described herein. As used herein, the term “topical formulation” refers to a formulation that may be applied directly to a part of the body. The term “formulation” is used herein to denote compositions of various ingredients in various weight ranges, in accordance with the present disclosure for use in personal or home care.

“Personal care” means and comprises any cosmetic, hygienic, toiletry and topical care products including, without limitation, leave-on products (i.e., products that are left on keratinous substrates after application); rinse-off products (i.e., products that are washed or rinsed from keratinous substrates during or within a few minutes of application); shampoos; hair curling and hair straightening products; hair style maintaining and hair conditioning products; lotions and creams for nails, hands, feet, face, scalp and/or body; hair dye; face and body makeup; nail care products; astringents; deodorants; antiperspirants; anti-acne; antiaging; depilatories; colognes and perfumes; skin protective creams and lotions (such as sunscreens); skin and body cleansers; skin conditioners; skin toners; skin firming compositions; skin tanning and lightening compositions; liquid soaps; bar soaps; bath products; shaving products; and oral hygiene products (such as toothpastes, oral suspensions, and mouth care products).

The texture of such personal care formulations is not limited and may be, without limitation, a liquid, gel, spray, emulsion or blend (such as lotions and creams). Sprays can be non-pressurized aerosols delivered from manually pumped finger-actuated sprayers or can be pressurized aerosols such as mousse, spray, or foam forming formulation, where a chemical or gaseous propellant is used.

Formulations prepared using the a blend of modified starches and a 1,3-β-D-glucan disclosed herein have a white or pale white color that is generally considered to be aesthetically appealing. In some cases, the formulations of this disclosure may be further processed to make a colored end product. In such cases, the white color is beneficial because it will show up the additional pigment without influencing the final color.

Formulations containing the modified starch and hydrocolloid of the present disclosure may optionally contain additional ingredients to tailor the viscosity to the needs of the particular application. A skilled artisan will readily appreciate the wide range of additives available to suit this purpose.

It is understood that the personal care composition as described herein contains an active ingredient selected from the group consisting of antiperspirants, fragrances, and mixtures thereof.

The antiperspirant active can be selected from the group consisting of any of the known. antiperspirant active materials. These include, by way of example (and not of a limiting nature), aluminum chlorohydrate, aluminum chloride, aluminum sesquichlorohydrate, zirconyl hydroxychloride, aluminum-zirconium glycine complex (for example, aluminum zirconium trichlorohydrex gly, aluminum zirconium pentachlorohydrex gly, aluminum zirconium tetrachlorohydrex gly and aluminum zirconium octochlorohydrex gly), aluminum chlorohydrex ⋅PG, aluminum chlorohydrex PEG, aluminum dichlorohydrex PG, and aluminum dichlorohydrex PEG. The aluminum-containing materials can be commonly referred to as antiperspirant active aluminum salts.

Generally, the foregoing metal antiperspirant active materials are antiperspirant active metal salts. In the embodiments which are antiperspirant compositions according to the present invention, such compositions need not include aluminum containing metal salts, and can include other antiperspirant active materials, including other antiperspirant active metal salts. Generally, Category I active antiperspirant ingredients listed in the Food and Drug Administration's Monograph for antiperspirant drugs for over-the-counter human use can be used. In addition, any new drug, not listed in the Monograph, such as aluminum nitratohydrate and its combination with zirconyl hydroxychlorides and nitrides, or aluminum-stannous chlorohydrates, can be incorporated as an antiperspirant active ingredient in antiperspirant compositions

Particular types of antiperspirant actives include aluminum zirconium trichlorohydrex and aluminum zirconium tetrachlorohydrex either with or without glycine. A particular antiperspirant active is aluminum trichlorohydrex gly such as AZZ-902 SUF (from Reheis Inc., Berkley Heights, NJ) which has 98% of the particles less than 10 microns in size.

Antiperspirant actives can be incorporated into compositions according to the present invention in amounts in the range of 0.1-25% of the final composition, but the amount used will depend on the formulation of the composition. For example, at amounts in the lower end of the broader range (for example, 0.1-10% on an actives basis), a deodorant effect may be observed. At lower levels the antiperspirant active material may not substantially reduce the flow of perspiration, but may reduce malodor, for example, by acting as an antimicrobial material. At amounts of 5-25% (on an actives basis) such as 15-25%, by weight, of the total weight of the composition, an antiperspirant effect may be observed.

Personal care compositions may further comprise a glycerol or polyglycerol. The glycol or polyglycol is selected from the group consisting of ethylene glycol, propylene glycol, 1,2-propanediol, diethylene glycol, triethylene glycol, tetraethylene glycol, dipropylene glycol, tripropylene glycol, methyl propanediol, 1,6-hexanediol, 1,3-butanediol, 1,4-butanediol, PEG-4 through PEG-100, PPG-9 through PPG-34, pentylene glycol, neopentyl glycol, trimethylpropanediol, 1,4-cyclohexanedimethanol, 2,2-dimethyl-1,3-propanediol, 2,2,4,4-tetramethyl-1,3-cyclobutanediol, and mixtures thereof. More particular examples of the glycol component include one or more members of the group consisting of propylene glycol, dipropylene glycol, tripropylene glycol, 2-methyl-1,3-propanediol, methyl propylene glycol, low molecular weight (less than 600) polyethylene glycol, low molecular weight (less than 600) polypropylene glycols, and mixtures of any of the foregoing. Propylene glycol is of particular interest because the antiperspirant active may be more soluble in this type of glycol. Tripropylene glycol has lower irritancy, but the antiperspirant active may not be as soluble in this glycol. Mixtures of glycols may be used to balance these desirable properties.

As used herein the terms “fragrance” and “perfume” have the same intended meaning. Fragrances and perfumes are materials added to provide users the experience of a scent. They may be any material that provide such a scent and are typically derived from herbal, floral, or other natural sourced oils and materials. They maybe natural or synthetic and are well known to those of skill in the art. Aspects disclosed herein incorporate desirable scents through inclusion of fragrances or perfumes and perfume raw materials in perfume delivery systems. Certain perfume delivery systems, methods of making certain perfume delivery systems, and the uses of such perfume delivery systems are disclosed in U.S. Pre-Grant Publication No. 2007/0275866 A1. The perfumes and perfume raw materials previously disclosed can be used in such perfume delivery systems. Such perfume delivery systems include: polymer-assisted delivery (PAD), molecule-assisted delivery (MAD), fiber-assisted deliver (FAD), amine-assisted delivery (AAD), cyclodextrin delivery system (CD), starch encapsulated accord (SEA), inorganic carrier delivery system (ZIC), and Pro-Perfume (PP). Examples of these perfume delivery systems are well known in the art.

In aspects disclosed herein, the fragrances/perfumes disclosed herein, and stereoisomers thereof, are suitable for use, in perfume delivery systems at levels, based on total perfume delivery system weight, of from 0.001% to about 50%, from 0.005% to 30%, from 0.01% to about 10%, from 0.025% to about 5%, or even from 0.025% to about 1%.

In one aspect, the perfume delivery systems disclosed herein are suitable for use in personal care compositions at levels, based on total personal care composition weight, from about 0.001% to about 20%, from about 0.01% to about 10%, from about 0.05% to about 5%, from about 0.1% to about 0.5%. The inclusion rate will depend on specific fragrance(s) chosen and is determined in the discretion of the skilled artisan.

It is understood that the personal care composition as described herein can optionally contain other components, including one or more beneficial agents.

In one embodiment, the beneficial agent comprises one or more compounds selected from emollients, moisturizers, conditioners, skin conditioners, vitamins, pro-vitamins, antioxidants, free-radical scavengers, abrasives, dyes, hair coloring agents, bleaching agents, UV absorbers, anti-UV agents, antibacterial agents, antifungal agents, melanin regulators, tanning accelerators, depigmenting agents, skin lightening agents, skin-coloring agents, lipo-regulators, weight-reduction agents, anti-acne agents, anti-ageing agents, anti-wrinkle agents, refreshing agents, vascular-protection agents, agents for the reduction of dandruff, seborrheic dermatitis, or psoriasis, shale oil and derivatives thereof, anti-psoriasis agents, corticosteroids, depilating agents, agents for combating hair loss, reducing agents for permanent-waving, and essential oils.

In one embodiment, the personal care composition further comprises one or more water insoluble or partially water soluble components. Such components may be in the form of a solid, a liquid, or a gas and may comprise one or more materials selected from water insoluble or partially water soluble additional agents including by not limited to; emollients, conditioners, moisturizers, vitamins, vitamin derivatives, moisturizing beads, natural or synthetic abrasives, such as polyoxyethylene beads, abrasives, and water insoluble or partially water soluble chemically stable appearance modifying additives such as, for example, pigments, opacifying agents, colored or reflective particles or beads such as particles of mica, titanium dioxide, or glycol stearate.

Formulations of the present disclosure may optionally contain at least one further ingredient chosen from the group consisting of preservative, salt, vitamin, emulsifier, texturizer, nutrient, micronutrient, sugar, protein, polysaccharide, polyol, glucose, sucrose, glycerol, sorbitol, pH adjusters, emollients, dyes, pigments, skin actives, waxes, or silicones.

In some aspects of the present disclosure silicone(s) is/are present in the formulation in an amount between 1 and 20% or between 1 and 6%. In some aspects, the silicone is cyclomethicone and is present between 1 and 6%.

In some aspects, the personal care formulation contains between 3% and 4% of the texturizer, between 0.2% and 0.6% of a 1,3-β-D-glucan, and between 2% and 4% of a silicone.

In some aspects, the personal care formulation contains between 3% and 4% of the texturizer, between 0.2% and 0.6% of a 1,3-β-D-glucan, and between 2% and 4% of a silicone; wherein the texturizer is a blend of HP starch and nOSA starch, the 1,3-β-D-glucan is a sclaroglucan, and the silicone is cyclomethacone.

Formulations of the present disclosure may have a wide range of pH values. Aspects of this disclosure include formulations having pH between 3-11, or between 4-8, or between 4-7.

In some embodiments of the present disclosure the personal care formulation is a deodorant or antiperspirant. In some embodiments the deodorant or antiperspirant is packaged as a roll-on.

Examples of suitable roll-on dispensers include those described in U.S. Design Pat. No. 402,550 to Poisson; U.S. Pat. No. 6,132,126 to Sheffer et al (an adjustable applicator); U.S. Pat. No. 4,030,844 to Lench et al; U.S. Pat. No. 4,021,125 to Berghahn et al; U.S. Pat. No. 4,033,700 to Spatz; U.S. Pat. No. 5,553,957 to Dornbusch et al; WO 00/64302 to Hindustan Lever Ltd.; and WO 01/03541 to Chang; all of which are incorporated by reference herein to the extent they describe roll-on dispensers.

Preparation of antiperspirants and deodorant formulations is well known in the art. Typically, a formulation is prepared from the following components: water, thickeners, conditioning agents, preservatives, fragrances, antiperspirant actives and other ingredients.

Water is the primary ingredient in all roll-on products, it typically makes up about 50 to 80% of the entire formula. It helps dilute the ingredients, makes the formula easier to spread, and reduces irritation. It also keeps the formula inexpensive.

Thickeners are used to adjust the viscosity and texture of the formulation. To some extent the secondary detergents make shampoo formulations thicker. Simply adding salt can also increase shampoo thickness. However, other materials are also used to increase the viscosity such as methylcellulose which is a cellulosic polymer or carbomer which is a synthetic polymer.

Any formula that contains water has the potential to be contaminated by bacteria and other microbes. For this reason, preservatives are added to prevent such growth. Two of the most common preservatives are DMDM Hydantoin and methylparaben. For personal care products ingredients like sodium benzoate, benzyl alcohol and phenoxyethanol are also used.

### Viscosity Study

A surprising viscosity synergy was observed with formulations disclosed herein when anti-perspirant actives such an active aluminium salt is added. In some aspects aluminum chlorohydrate (ACH) is added. Typically, and almost universally, formulations experience a drop in the overall viscosity of the formulation over time. Therefore, to maintain sufficient viscosity additional texturizers may be required to be added into the formulation. This is results in formulations with higher costs, more complexity, and dilution of other ingredients. An experiment was designed based on the Scheffe Simplex Centroid+Axial Design method. Three experimental details were investigated. The amount of starch blend (Star Design Power (SDP) from Cargill) was varied between 3% and 6%. The amount of cyclomethicone was varied between 0 and 6%, and the amount of sclaroglucan (CS11 QD from Cargill) was varied between 0 and 1.2%. 10 samples were prepared according to the following table. The samples were repeated with inclusion of 20% ACH to the formula.

TABLE 1

| EXPERIMENTAL DESIGN | | | | 3 COMPONENTS | | | |
|---|---|---|---|---|---|---|---|
| 3 COMPONENTS (theory) | | | | (actual in Wt % in Formula) | | | |
| # | SDP | CS11 QD | CM | # | SDP | CS11 QD | CM |
| 1 | 1 | 0 | 0 | 1 | 6 | 0 | 0 |
| 2 | 0 | 1 | 0 | 2 | 3 | 1.2 | 0 |
| 3 | 0 | 0 | 1 | 3 | 3 | 0 | 6 |
| 4 | 0.5 | 0.5 | 0 | 4 | 4.5 | 0.6 | 0 |
| 5 | 0.5 | 0 | 0.5 | 5 | 4.5 | 0 | 3 |
| 6 | 0 | 0.5 | 0.5 | 6 | 3 | 0.6 | 3 |
| 7 | 0.33 | 0.33 | 0.33 | 7 | 4 | 0.4 | 2 |
| 8 | 0.667 | 0.167 | 0.167 | 8 | 5 | 0.2 | 1 |
| 9 | 0.167 | 0.667 | 0.167 | 9 | 3.5 | 0.8 | 1 |
| 10 | 0.167 | 0.167 | 0.667 | 10 | 3.5 | 0.2 | 4 |

SDP is a Star Design Power from Cargill, CS-11 is sclaroglucan from Cargill and CM is cyclomethicone. The remainder of all formulations is water.

Viscosity can be evaluated with using a Brookfield DV3T instrument at (20 rpm, 25° C., 5#). Viscosity was evaluated on days, 1, 14, and 30. Very surprisingly, it was observed that the viscosity drops for formulations without ACH.

Table 2 shows a clear and dramatic viscosity drop of samples without ACH after 14 days and 30 days at room temperature. All samples show a reduction in viscosity of between 30% and 60%, with most samples exceeding 50%.

TABLE 2

| | 14 d RT | 30 d RT |
|---|---|---|
| #1 | −57.37 | −57.64 |
| #2 | −32.89 | −42.65 |
| #3* | — | — |
| #4 | −41.94 | −57.77 |
| #5 | −55.56 | −59.72 |
| #6 | −39.69 | −53.11 |
| #7 | −44.87 | −54.96 |
| #8 | −48.61 | −57.52 |
| #9 | −36.36 | −42.18 |
| #10 | −57.28 | −64.23 |

*Sample 3 was not stable upon preparation

Samples containing ACH were prepared as a comparison to the controls of Table 2. Very surprisingly, as seen in Table 3, the inclusion of 20% ACH to the formulas led to a significant retention of viscosity. All samples in Table 3 below show an increase of viscosity after 14 days as compared to an average >40% reduction as in the control. Even after 1 month the loss of viscosity of the samples averages less than 20% compared to over 50% in the controls.

TABLE 3

| | 14 d RT | 30 d RT |
|---|---|---|
| #1 | 26.05 | −23.10 |
| #2 | 2.26 | −1.07 |
| #3* | — | — |
| #4 | 13.59 | −19.01 |
| #5 | 27.13 | −26.60 |
| #6 | 12.40 | −9.81 |
| #7 | 5.10 | −15.25 |
| #8 | 22.56 | −19.70 |
| #9 | 0.00 | −16.46 |
| #10 | 9.85 | −11.54 |

*Sample 3 was not stable upon preparation

### Methods of Measurement

The speed of film formation can be monitored or estimated using dynamic viscoelastic measurements. These measurements were carried out using a stress controlled rheometer (MCR 302, Anton Paar Physica) equipped with a plate-and-plate geometry with both upper and lower surface crosshatched, and a peltier temperature controller. The principle of this approach consists in using the log normalized complex shear modulus as a function of time. All formulations were evaluated at 37° C. and tested in triplicate. Thus, the kinetic of the film formation was estimated using the slope from the linear part of the curve of the normalized complex shear modulus. The lower the slope, the faster the film forming speed.

Explanations of abbreviations and terms used in this disclosure are provided to assist in comprehending and practicing the invention.

All ratios of formulation components refer to percentage by weight (wt %), unless otherwise specified.

All parameter ranges disclosed include the end-points and all values in between, unless otherwise specified.

Representative features are set out in the following description, which stand alone or may be combined, in any combination, with one or more features disclosed elsewhere in the description and/or drawings of the specification.

When used in this specification and claims, the terms “comprises” and “comprising” and variations thereof mean that the specified features, steps or integers are included. The terms are not to be interpreted to exclude the presence of other features, steps or components.

### EXAMPLES

TABLE 4

| INCI Name | Trade Name | Ex 1 | Ex 2 | Ex 3 | Ex 4 |
|---|---|---|---|---|---|
| Scleroglucan | Actigum CS11 | 0.5 | 1.0 | 2.0 | 4.0 |
| | Cargill Inc | | | | |
| Starch Blend | Star Design Power | 3 | 4 | 3.5 | 3.5 |
| | Cargill Inc | | | | |
| Cyclopentasiloxane | DC345 Dow | 3 | 2 | 4 | 3 |
| (cyclomethicone) | Corning | | | | |
| Aluminum | Achieve L1050 | 20 | 20 | 20 | 20 |
| chlorohydrate | Gouldbransen | | | | |
| Water | | QS | QS | QS | QS |

The examples are prepared by the following method.

Water was stirred while the starch blend and scleroglucan are added. The mixture was then homogenized at 5000 rpms for approximately 10 minutes. Stirring was slowed for addition of the cyclomethicone and aluminium chlorohydrate. This mixture was then homogenized at 5000 rpms for approximately 1-2 minutes to create the final product.

### Competitive Benchmark Products

NIVEA® DRY COMFORT (BENCHMARK 1): Water, Aluminum Chlorohydrate, PPG-5 Stearyl Ether, Steareth-2, Steareth-21, Parfum, Aluminum Sesquichlorohydrate, Magnesium Aluminum Silicate, Persea Gratissima Oil, Trisodium EDTA, BHT, Linalool, Limonene, Benzyl Alcohol, Alpha-Isomethyl Ionone, Geraniol, Citronellol, Benzyl Salicylate.

DOVE® INVISIBLE DRY (BENCHMARK 2): Water, Aluminum Zirconium Pentachlorohydrate, Glycerin, Helianthus Annuus Seed Oil, Steareth-2, Parfum, Steareth-20, Tocopheryl Acetate, Citric Acid, Alpha-Isomethyl Ionone, Benzyl Alcohol, Benzyl Salicylate, Cinnamyl Alcohol, Citronellol, Geraniol, Hexyl Cinnamal, Hydroxycitronellal, Limonene, Linalool.

### Sensory Evaluation

A sensory panel of 13 participants, 7 male and 6 female, were used to evaluate the sensory aspects of the examples in comparison to the benchmarks. In a randomized blind test, application of 25 μl of test sample was made to the inner arm and spread to a circle of 4 cm in diameter (2 μl/cm²).

All 4 examples of the present disclosure were perceived by the panel to dry substantially faster (in approximate 1.5 minutes) than the benchmarks (approximately 2 min). This 25% improvement is highly significant when considered in the context of use by the consumer.

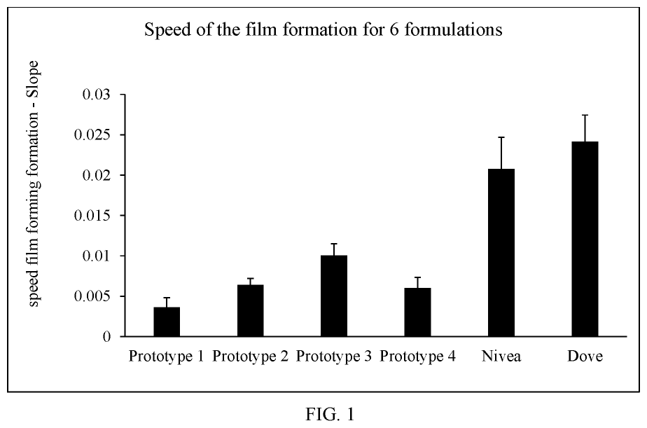

All 4 Examples/Prototypes were evaluated to the speed of film formation. The results are shown in FIG. 1. All of the Prototypes were dramatically more efficient in film formation than the commercial benchmarks. This result is a significant advantage to the ultimate consumer and user of the formulations disclosed herein.

We claim:

1. A personal care formulation for masking odor or reducing perspiration comprising a texturizer, a 1,3-β-D-glucan, and an active ingredient wherein: the texturizer is a mixture of hydroxypropylated starch and a nOSA starch; and the active ingredient is selected from the group consisting of fragrances and anti-perspirant actives or a combination thereof.

2. The personal care formulation of claim 1 wherein the 1,3-β-D-glucan is a sclaroglucan and the active ingredient is an antiperspirant active aluminum salt.

3. The personal care formulation of claim 2 wherein the sclaroglucan is present at 0.25% to 5% and the texturizer at 2% to 5% by weight of the composition.

4. The personal care formulation of claim 2 wherein the texturizer comprises hydroxypropylated starch in an amount of between 5-25% and, nOSA starch in an amount of in an amount of 75-95% by weight.

5. The personal care formulation of claim 3 wherein the antiperspirant active aluminum salt is present in the formulation at 15%-25% by weight.

6. The personal care formulation of claim 5 further comprising a silicone.

7. The personal care formulation of claim 6 where the silicone is cyclomethacone.

8. The personal care formulation of claim 7 that is a roll-on antiperspirant or deodorant.

9. The personal care formulation of claim 1 wherein the active ingredient is an anti-perspirant active.

10. The personal care formulation of claim 9 wherein the anti-perspirant active is an aluminum salt.
